(19) United States
(12) Patent Application Publication
(10) Pub. No.: US 2006/0259328 A1
(43) Pub. Date: Nov. 16, 2006

(54) Wireless Non-Invasive Analyte Measurement Device

(75) Inventors: John F. Burd, San Diego, CA (US); Paul Williams, San Diego, CA (US)

Correspondence Address: PROCOPIO, CORY, HARGREAVES & SAVITCH LLP, 530 B STREET, SUITE 2100, SAN DIEGO, CA, 92101, US

(73) Assignee: OCULIR, INC., San Diego, CA (US)

(21) Appl. No.: 11/460,145

(22) Filed: Jul. 26, 2006

Related U.S. Application Data
(63) Continuation-in-part of application No. 11/122,472, filed on May 5, 2005.
(63) Continuation of application No. 10/824,214, filed on Apr. 14, 2004, now Pat. No. 6,975,892.
(60) Provisional application No. 60/513,396, filed on Oct. 21, 2003.

(51) Int. Cl. G06Q 50/00; G06Q 10/00; A61B 5/00

(52) U.S. Cl. 705/2; 600/300

(57) ABSTRACT

Systems and method for tracking measurements from a wireless measurement device are provided. A tracking server is in communication with wireless measurement devices deployed in the field and periodically receives usage reports from the measurement devices to ensure timely measurements by the user and to monitor usage of the measurement devices to ensure compliance. The wireless measurement device is configured to measure one or more analyte levels or concentrations, presence, and/or absence of one or more analytes of the user. The fact of the measurement and the results of the measurement are provided to the tracking server via a wireless communication network. The tracking server stores the information for historical tracking of measurement data usage data. The tracking server may send alerts to a predetermined list of people to notify them when the user is not using the measurement device or when the number of authorized uses needs to be replenished.

### RELATED APPLICATION

This application is a continuation-in-part application of U.S. patent application Ser. No. 11/122,472, filed May 5, 2005, which is a continuation application of U.S. patent application Ser. No. 10/824,214, filed Apr. 14, 2004, and claims the benefit of prior provisional application 60/513,396, filed on Oct. 21, 2003 under 35 U.S.C. 119(e). This application claims the benefit of these prior applications and these applications are incorporated by reference herein as though set forth in full.

### BACKGROUND

1. Field of the Invention

The present invention generally relates to mobile medical diagnostic measurement devices and more particularly relates to operational control of mobile medical diagnostic measurement device via a wireless communication network.

2. Related Art

Diabetes remains one of the most serious and under-treated diseases facing the worldwide healthcare system. Diabetes is a chronic disease where the body fails to maintain normal levels of glucose in the bloodstream. It is now the fifth leading cause of death from disease in the U.S. today and accounts for about 15% of the entire healthcare budget. People with diabetes are classified into two groups: Type 1 (formerly known as “juvenile onset” or “insulin dependent” diabetes, that are required to take insulin to maintain life) and Type 2 (formerly known as “adult onset” or “non-insulin dependent,” that may require insulin but may sometimes be treated by diet and oral hypoglycemic drugs). In both cases, without dedicated and regular blood glucose measurement, all patients face the possibility of the complications of diabetes that include cardiovascular disease, kidney failure, blindness, amputation of limbs and premature death.

The number of cases of diabetes in the U.S. has jumped 40% in the last decade. This high rate of growth is believed to be due to a combination of genetic and lifestyle origins that appear to be a long-term trend, including obesity and poor diet. The American Diabetes Association (ADA) and others estimate that about 17 million Americans and over 150 million people worldwide have diabetes, and it is estimated that up to 40% of these people are currently undiagnosed [American Diabetes Association, “Facts & Figures”].

Diabetes must be “controlled” in order to delay the onset of the disease complications. Therefore, it is essential for people with diabetes to measure their blood glucose levels several times per day in an attempt to keep their glucose levels within the normal range (80 to 126 mg/dL). These glucose measurements are used to determine the amount of insulin or alternative treatments necessary to bring the glucose level to within target limits. Self-Monitoring of Blood Glucose (SMBG) is an ongoing process repeated multiple times per day for the rest of the patient's lifetime.

All currently Food and Drug Administration (“FDA”) approved invasive or “less-invasive” (blood taken from the arm or other non-fingertip site) glucose monitoring products currently on the market require the drawing of blood in order to make a quantitative measurement of blood glucose. The ongoing and frequent measurement requirements (1 to possibly 10 times per day) presents all diabetic patients with pain, skin trauma, inconvenience, and infection risk resulting in a general reluctance to frequently perform the critical measurements necessary for selecting the appropriate insulin dose or other therapy.

These current product drawbacks have led to a poor rate of patient compliance. Among Type 1 diabetics, 39% measure their glucose levels less than once per day and 21% do not monitor their glucose at all. Among Type 2 diabetics who take insulin, only 26% monitor at least once per day and 47% do not monitor at all. Over 75% of non-insulin-taking Type 2 diabetics never monitor their glucose levels [Roper Starch Worldwide Survey]. Of 1,186 diabetics surveyed, 91% showed interest in a non-invasive glucose monitor. As such, there is both a tremendous interest and clinical need for a non-invasive glucose measurement device. A further need exists for systems and methods to track glucose measurements from a non-invasive glucose measurement device to ensure timely use and for monitoring the use of such a device to ensure compliance.

### SUMMARY

Accordingly, the present invention provides systems and methods for tracking the measurements from a wireless enabled non-invasive analyte measurement (“NAM device”) device to ensure timely use and for monitoring the use of such wireless devices to ensure compliance. The wireless enabled NAM device is configured to interrogate a body surface, for example, the eye, of a user with an electromagnetic radiation signal and determine the user's level of one or more analytes, for example, glucose. The occurrence of the measurement and the results of the measurement are provided to a tracking server via a network. The server stores the information and updates any individual parameters based on the new information. If the tracking server does not receive the measurement information from the NAM device, then an alert may be sent out to a predetermined list of people to notify them that the user is not checking glucose levels. If the tracking server determines that the user's glucose level should be tracked, a recurring monitor program can be provided.

Other features and advantages of the present invention will become more readily apparent to those of ordinary skill in the art after reviewing the following detailed description and accompanying drawings.

### BRIEF DESCRIPTION OF THE DRAWINGS

The details of the present invention, both as to its structure and operation, may be gleaned in part by study of the accompanying drawings, in which like reference numerals refer to like parts, and in which:

FIG. 1 is a network diagram illustrating an example system for use of wireless enabled analyte measurement devices according to an embodiment of the present invention;

FIG. 2 is a block diagram illustrating an example wireless non-invasive analyte measurement device in operation according to an embodiment of the present invention;

FIG. 3 is a block diagram illustrating an example tracking server according to an embodiment of the present invention;

FIG. 4 is a block diagram illustrating an example user profile on a tracking server according to an embodiment of the present invention;

FIG. 5 is a block diagram illustrating an example measurement device profile on a tracking server according to an embodiment of the present invention;

FIG. 6 is a flow diagram illustrating an example process for tracking measurements by a wireless non-invasive analyte measurement device according to an embodiment of the present invention;

FIG. 7 is a flow diagram illustrating an example process for monitoring the user of a wireless non-invasive analyte measurement device according to an embodiment of the present invention; and

FIG. 8 is a block diagram illustrating an example wireless communication device that may be used in connection with various embodiments described herein; and

FIG. 9 is a block diagram illustrating an example computer system that may be used in connection with various embodiments described herein.

### DETAILED DESCRIPTION

Certain embodiments as disclosed herein provide for a wireless enabled non-invasive analyte measurement device (“NAM device”) that communicates with a tracking server via a wireless communication network. The wireless NAM device provides measurement data to the tracking server where the data is maintained and analyzed to monitor usage and account status. For example, one method as disclosed herein allows for the tracking server to authorize a wireless NAM device for a certain number of measurements and then track the number of measurements taken by the wireless NAM device. When the authorized number of uses reaches a certain threshold, the tracking server can notify the user or another party so that the number of authorized uses can be replenished.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

Additionally, in the context of this application, the term “analyte” as used herein describes any particular substance or chemical constituent to be measured. Analyte may also include any substance in the tissue of a subject, in a biological fluid (for example, blood, interstitial fluid, cerebral spinal fluid, lymph fluid or urine), or is present in air that was in contact with or exhaled by a subject, which demonstrates an electromagnetic radiation signature, for example, infrared. Analyte may also include any substance which is foreign to or not normally present in the body of the subject. Analytes can include naturally occurring substances, artificial substances, metabolites, and/or reaction products. In some embodiments, the analyte for measurement by the devices and methods described herein is glucose. However, other analytes are contemplated as well, including, but not limited to, metabolic compounds or substances, carbohydrates such as sugars including glucose, proteins, glycated proteins, fructosamine, hemoglobin Alc, peptides, amino acids, fats, fatty acids, triglycerides, polysaccharides, alcohols including ethanol, toxins, hormones, vitamins, bacteria-related substances, fungus-related substances, virus-related substances, parasite-related substances, pharmaceutical or non-pharmaceutical compounds, substances, pro-drugs or drugs, and any precursor, metabolite, degradation product or surrogate marker of any of the foregoing. Other analytes are contemplated as well, including, but not limited, to acarboxyprothrombin; acylcamitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-ethylchloroquine; dehydroepiandrosterone sulfate; nucleic acids (deoxyribonucleic acids and ribonucleic acids including native and variant sequences related to acetylator polymorphism, alcohol dehydrogenase, alpha 1-antitrypsin, cystic fibrosis, Down's syndrome, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, PKU, Plasmodium vivax, sexual differentiation, 21-hydroxylase); 21-deoxycortisol; desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1,); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); neurotransmitters (such as glutamate, GABA, dopamine, serotonin), opioid neurotransmitters (such as endorphins, and dynorphins), neurokinins (such as substance P); succinylacetone; sulfadoxine; theophylline; thyrotropin (TSH); thyroxine (T4); thyroxine-binding globulin; trace elements; transferrin; UDP-galactose-4-epimerase; urea; prokaryotic and eukaryotic cell-surface antigens; peptidoglycans; lipopolysaccharide; uroporphyrinogen I synthase; vitamin A; white blood cells; and zinc protoporphyrin. Salts naturally occurring in blood or interstitial fluids can also constitute analytes in certain embodiments. The analyte can be naturally present in the biological fluid, for example, a metabolic product, an antigen, an antibody, and the like. Alternatively, the analyte can be introduced into the body, for example, a contrast agent for imaging, a radioisotope, a chemical agent, a fluorocarbon-based synthetic blood, or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); tricyclic antidepressants, benzodiazepines, acetaminophen (paracetamol, APAP), aspirin, methadone, hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (5HIAA).

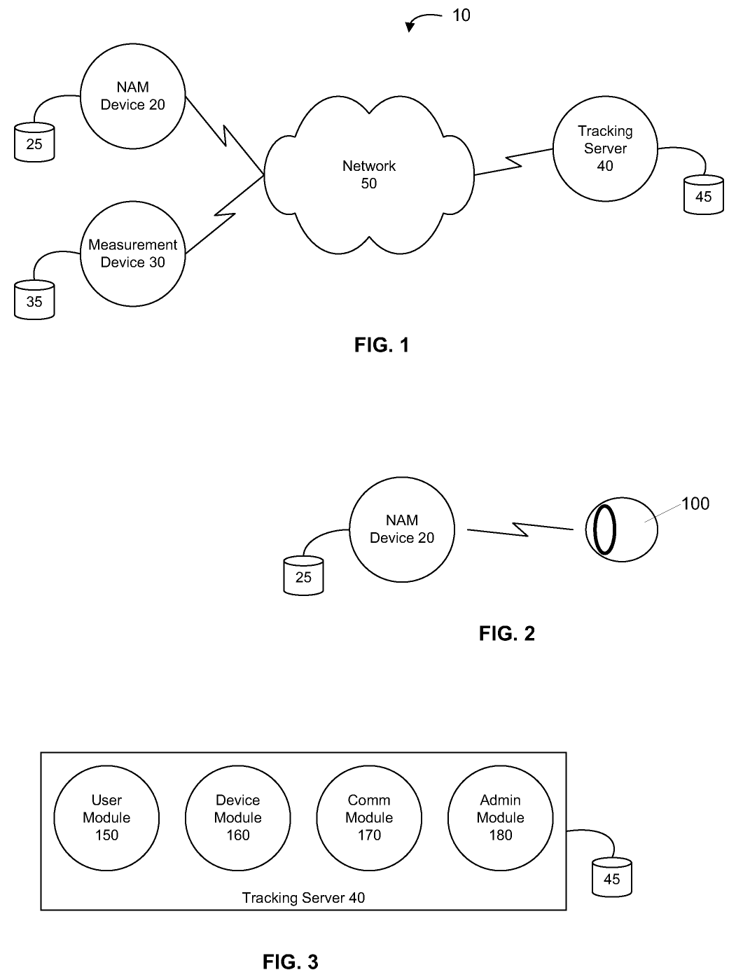

FIG. 1 is a network diagram illustrating an example system 10 for use of wireless enabled analyte measurement devices according to an embodiment of the present invention. In the illustrated embodiment, the system 10 comprises NAM device 20 and measurement device 30. As used herein, the “measurement device 30” may be a NAM device and/or a different type of measurement device. The NAM device 20 is configured with data storage area 25 and the measurement device 30 is configured with data storage area 35. The system 10 may include more or fewer NAM devices 20 and/or measurement devices 30. The system 10 also includes a tracking server 40 that is communicatively coupled with the NAM device 20 and the measurement device 30 via a network 50. The tracking server 40 is also configured with a data storage area 45.

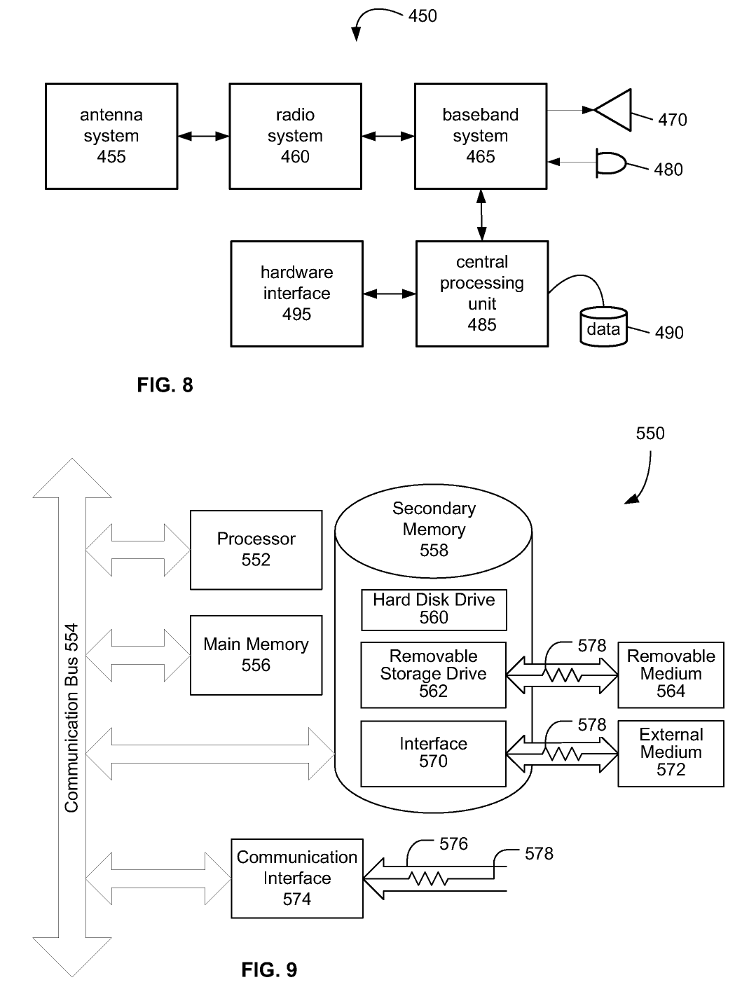

The NAM device 20 is a non-invasive analyte measurement device that is configured for communication over network 50. The NAM 20 can be integrated into any of a variety of types of wired or wireless communication devices including a personal digital assistant (“PDA”), cellular telephone, handheld gaming device, personal computer, laptop computer, or other device that is capable of communication with the tracking server 40 over the network 50. A general purpose wireless communication device is described later with respect to FIG. 8 and a general purpose computer device is described later with respect to FIG. 9.

The measurement device 30 can be any of a variety of measurement device types including invasive measurement devices and the like. The measurement device 30 is also configured for communication with the tracking server 40 over the network 50. The measurement device 30 can be integral to or combined with any sort of wired or wireless communication device.

To simplify the detailed description, the embodiment described will focus on the wireless NAM device 20 and its use in the system 10, but it should be understood that the breadth of the invention contemplates the use of additional and/or other types of measurement devices that are communicatively coupled with the tracking server 40 via a wired or wireless network.

The tracking server 40 can be any of a variety of computing devices and platforms that are capable of communication with NAM device 20 or measurement device 30 over the network 50. The tracking server 40 is configured with a data storage area 45.

The data storage areas 25, 35, and 45 can be any sort of internal or external, fixed or removable memory device and may include both persistent and volatile memories. The function of the data storage area 35 is to maintain data for long term storage and also to provide efficient and fast access to instructions for applications or modules that are executed by the their respective devices.

The network 50 may be any of a variety of network types and topologies and any combination of such types and topologies. For example, the network 50 may comprise a plurality of networks including private, public, circuit switched, packet switched, personal area networks (“PAN”), local area networks (“LAN”), wide area networks (“WAN”), metropolitan area networks (“MAN”), or any combination of the these. Network 50 may also include that particular combination of networks ubiquitously known as the Internet.

FIG. 2 is a block diagram illustrating an example wireless NAM device 20 in operation according to an embodiment of the present invention. In the illustrated embodiment, the NAM device 20 interrogates a body surface of the subject, for example the eye 100. Advantageously, the interrogation can be accomplished using electrogmagnetic signals, and more advantageously, infrared (“IR”) signals, such that the measurement is taken non-invasively. As a result of the interrogation, the NAM device 20 measures one or more analyte levels (including the absence thereof) for the subject and the measured concentration, presence, and/or absence of one or more analytes can be stored in the data storage area 25. Measurements can be taken periodically by the NAM device 20 such that the data for multiple interrogations can be stored in the data storage area 25. These measurements and other data can be sent to the tracking server 40 so that information regarding the measurements themselves and additional information including the number of uses of the NAM device 20 can be tracked by the tracking server 40.

FIG. 3 is a block diagram illustrating an example tracking server 40 according to an embodiment of the present invention. In the illustrated embodiment, the tracking server 40 is configured with a data storage area 45 and the tracking server 40 comprises a user module 150, a device module 160, a communication module 170, and an administrative module 180. Advantageously, the tracking server 40 is configured to monitor and track users/subjects and their use of the NAM device 20 as well as the measurements and other information collected by the NAM device 20. The tracking server 40 is also configured to track NAM devices 20 and other measurement devices 30 to authorize the devices for continued use.

The user module 150 is configured to maintain a plurality of user profiles and information related to the particular user/subject. For example, this information may include historical measurement information for the user. A user may also be associated with a particular NAM device 20 so that measurements that are provided to the tracking server 40 from a particular NAM device 20 are associated with the appropriate user. The user module 150 may also maintain billing records and other information about the user that is beneficial in continued operation of the NAM device 20 and its use in the overall system.

The device module 160 is configured to maintain a plurality of device profiles and information related to a plurality of different NAM devices 20 or other measurement device types. For example, this information may include details about the characteristics and capabilities of the particular device such as the various types of analytes that it measures, how the device measures the analytes (e.g., IR versus strip), the wireless communication abilities of the device, the amount and type of information compiled into a measurement report by the device, and other beneficial information.

The device module 160 is also configured to track individual devices (regardless of type) and coordinate the exchange of information with the individual devices. For example, the device module 160 may inform the device about certain operational characteristics for the device such as the recommended frequency of measurements and the type of information that should be collected and returned to the tracking server 40. Other operational characteristics may include the number of authorized measurements the device may take pursuant to the account status of the user of the device.

In one embodiment, the user of the device may be an individual who employs the NAM device 20 to measure only the user's own analyte levels (e.g., the concentration, presence, and/or absence of one or more analytes). In an alternative embodiment, the user of the device may be an entity such as a quick care facility or the like that employs the NAM device 20 to measure analyte levels of patients or customers.

The communication module 170 is configured to manage communications between the tracking server 40 and the various NAM devices 20 and other measurement devices 30 deployed in the field. Such communications may travel over a wired or wireless network such as the network previously described with respect to FIG. 1. The communication module 170 is configured to send instructions to and receive data from the various measurement devices 30 deployed in the field and coordinate communications between the user module 150 and the measurement devices 30, between the device module 160 and the measurement devices 30, and the admin module 180 and the measurement devices 30. The communication module 170 may also be employed to handle communications between the various modules of the tracking server 40.

The admin module 180 is configured to handle the administrative functions of the tracking server 40. These functions may include management of the data stored in the data storage area 45 as well as credit card and other billing and financial aspects of the overall system. Admin module 180 may also work with communication module 170 to communicate with back-end financial data processors, credit card companies, etc. in order to accept and receive payments from end users to keep accounts active and authorize additional uses of the measurement devices 30 deployed in the field.

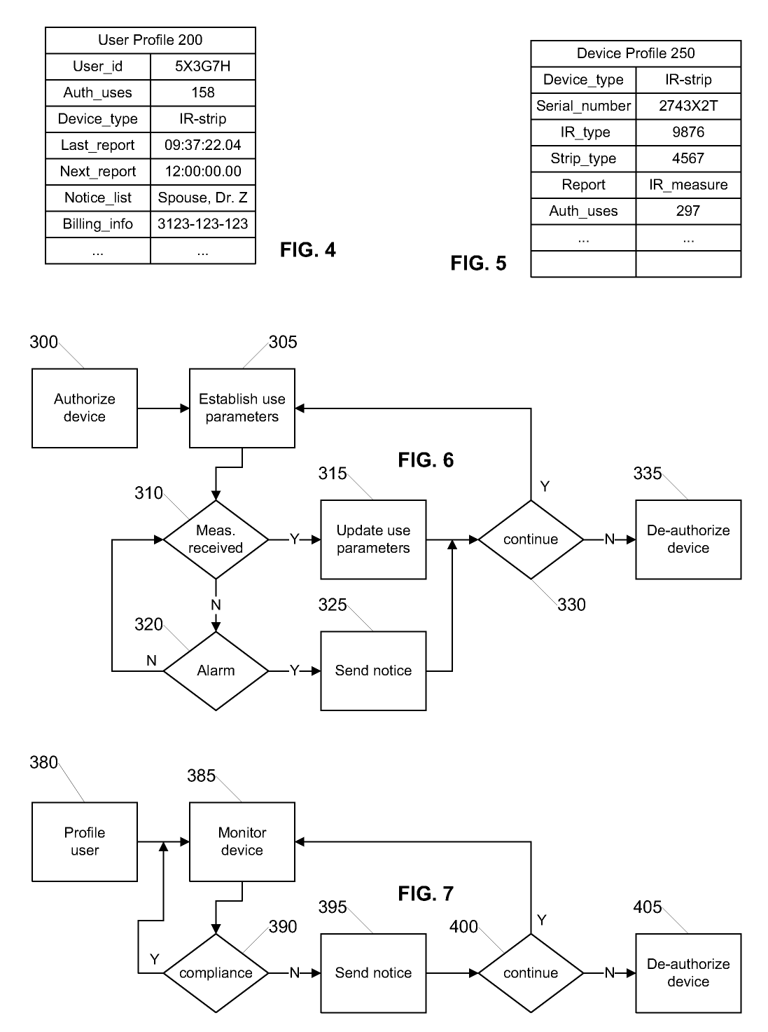

FIG. 4 is a block diagram illustrating an example user profile 200 on a tracking server according to an embodiment of the present invention. In the illustrated embodiment, the user profile 200 comprises fields for user identification, authorized number of uses, device type, last report time, next report time, notification list, and billing information. In one embodiment, the user identification preferably uniquely identifies the user on the tracking server, the authorized uses identifies the number of remaining authorized uses for the particular user, the type of measurement device 30 the user has, the last time that a measurement report was received for the user, the next time that a measurement report is expected for the user, a list of contact information for people that are to be notified when notification is required to prompt reauthorization of uses or to prompt use of the measurement device 30 to measure analyte levels (e.g., the concentration, presence, and/or absence of one or more analytes), and the billing information includes a credit card number and transaction related information that allows the tracking server 40 to receive payment for additional authorized uses as requested by the user. Other information may also be included in the user profile 200, as will be understood by those having skill in the art.

FIG. 5 is a block diagram illustrating an example measurement device profile 250 on a tracking server according to an embodiment of the present invention. In the illustrated embodiment, the device profile 250 comprises fields for device type, serial number of the device, non-invasive measurement type, invasive measurement type, measurement report type, and the number of authorized uses. In one embodiment, the device type identifies the general or specific category of the device, the serial number preferably uniquely identifies the specific device, the IR type identifies the specific type of non-invasive measurements that are collected by the device, the strip type identifies the specific type of invasive measurements that are collected by the device, the measurement report identifies the format of the measurement report that is generated by the device and perhaps the information that is included in such a report, and the authorized number of uses identifies the number or remaining authorized uses for that particular device.

FIG. 6 is a flow diagram illustrating an example process for tracking measurements by a wireless measurement device 30 according to an embodiment of the present invention. The illustrated process may be carried out by a measurement device 30 such as that previously described with respect to FIG. 1. Initially, in step 300 the tracking server 40 authorizes the device. This may be done by setting up an account on the tracking server 40 and purchasing a certain number of authorized uses. Advantageously, this process may be undertaken via a web browser interface that is administered by the aforementioned administrative module on the tracking server. The number of authorized uses is sent to the measurement device 30. Once the device is authorized and is aware of its number of authorized uses, in step 305 the user parameters are establishes. Use parameters may include the frequency of expected use, which may be determined by a medical professional, for example.

Next, in step 310 the tracking server 40 checks to see if a measurement report has been received. If the expect report (based on the use parameters) has been received, as determined in step 310, then the use parameters are updated in step 315. For example, the updating of the use parameters may include resetting the time (or range) when the next measurement report is due and adjusting the remaining number of authorized uses.

If, as determined in step 310 that a measurement report has not been received, in step 320 it is determined whether the lack of a measurement report should trigger an alarm. If no alarm should be sent then the process loops back to check for the next received measurement report. If, as determined in step 320, an alarm should be sent then in step 325 such notice is sent. In one embodiment, the notice may be sent to any number of people or entities in the notice list contained in the user profile for the particular user. Notice is preferably sent by electronic communication (fax, email, page, instant message, or the like) but may also be sent by phone or even mail, although the more rapid receipt of the notice is preferred.

After notice has been sent in step 325 or the use parameters have been updated in step 315, the tracking server 40 determines in step 330 whether the monitoring should continue. If monitoring is to continue, the process loops back to confirm that the use parameters are appropriately established. In an alternative embodiment, after the use parameters are updated the process may automatically return to determine when the next measurement report is received. If the monitoring is not to proceed, as determined in step 330, then the tracking server 40 de-authorizes the device in step 335. For example, if the number of remaining authorized uses for the device has depleted to zero and the user has not authorized the tracking server 40 to replenish the number of authorized uses, then the tracking of the particular device may be de-authorized.

FIG. 7 is a flow diagram illustrating an example process for monitoring the user of a wireless measurement device 30 according to an embodiment of the present invention. The illustrated process may be carried out by a measurement device 30 such as that previously described with respect to FIG. 1. Initially, in step 380 the tracking server 40 completes a profile for the particular user that is to be monitored. This may be accomplished, for example, by providing information via a web browser interface under the control of an administrative module on the tracking server. Once a user profile has been established, then monitoring the user by way of the user device begins, as shown in step 385. As the monitoring ensues, the tracking server 40 periodically checks in step 390 to determine if the user (by way of the device) remains in compliance with the account parameters established and included in the user profile. This may mean that the user is using the measurement device 30 to take analyte measurements at a predetermined frequency. This may also mean that the user has remaining authorized uses associated with the user's account on the tracking server.

If the user is in compliance, as determined in step 390, the process loops back to continue monitoring. If the user is not in compliance, then in step 395 a notice may be sent to a list of persons or entities in the user profile to signal that the user account is no longer in compliance. In one embodiment, non-compliance may be determined when the number of authorized uses reaches a certain threshold, even though authorized uses remain. That way, a notice can be sent that will inform the user that the number of authorized used needs to be replenished without risking that the number reaches zero. Non compliance may also be determined when the user's expected measurement reports are not received for a certain delinquency period.

After notice has been sent, if the user account remains in good standing (e.g., the notice was informational) then in step 400 the tracking server 40 determines that the account should continue to be monitored and the process loops back to step 385 for continued monitoring. If, as determined in step 400, the monitoring should not continue, then the tracking server 40 de-authorizes the device and discontinues monitoring. For example, if the user requests that monitoring be discontinued or if the number of authorized uses reaches zero and no instruction is provided by the user to replenish the number of authorized uses.

FIG. 8 is a block diagram illustrating an exemplary wireless communication device 450 that may be used in connection with the various embodiments described herein. For example, the wireless communication device 450 may be used in conjunction with the NAM device 20 or the measurement device 30 previously described with respect to FIG. 1. However, other wireless communication devices and/or architectures may also be used, as will be clear to those skilled in the art.

In the illustrated embodiment, wireless communication device 450 comprises an antenna system 455, a radio system 460, a baseband system 465, a speaker 470, a microphone 480, a central processing unit (“CPU”) 485, a data storage area 490, and a hardware interface 495. In the wireless communication device 450, radio frequency (“RF”) signals are transmitted and received over the air by the antenna system 455 under the management of the radio system 460.

In one embodiment, the antenna system 455 may comprise one or more antennae and one or more multiplexors (not shown) that perform a switching function to provide the antenna system 455 with transmit and receive signal paths. In the receive path, received RF signals can be coupled from a multiplexor to a low noise amplifier (not shown) that amplifies the received RF signal and sends the amplified signal to the radio system 460.

In alternative embodiments, the radio system 460 may comprise one or more radios that are configured to communication over various frequencies. In one embodiment, the radio system 460 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 460 to the baseband system 465.

If the received signal contains audio information, then baseband system 465 decodes the signal and converts it to an analog signal. Then the signal is amplified and sent to the speaker 470. The baseband system 465 also receives analog audio signals from the microphone 480. These analog audio signals are converted to digital signals and encoded by the baseband system 465. The baseband system 465 also codes the digital signals for transmission and generates a baseband transmit audio signal that is routed to the modulator portion of the radio system 460. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 455 where the signal is switched to the antenna port for transmission.

The baseband system 465 is also communicatively coupled with the central processing unit 485. The central processing unit 485 has access to a data storage area 490. The central processing unit 485 is preferably configured to execute instructions (i.e., computer programs or software) that can be stored in the data storage area 490. Computer programs can also be received from the baseband processor 465 and stored in the data storage area 490 or executed upon receipt. Such computer programs, when executed, enable the wireless communication device 450 to perform the various functions of the present invention as previously described. For example, data storage area 490 may include various software modules (not shown) that were previously described with respect to FIG. 3.

In this description, the term “computer readable medium” is used to refer to any media used to provide executable instructions (e.g., software and computer programs) to the wireless communication device 450 for execution by the central processing unit 485. Examples of these media include the data storage area 490, microphone 470 (via the baseband system 465), antenna system 455 (also via the baseband system 465), and hardware interface 495. These computer readable mediums are means for providing executable code, programming instructions, and software to the wireless communication device 450. The executable code, programming instructions, and software, when executed by the central processing unit 485, preferably cause the central processing unit 485 to perform the inventive features and functions previously described herein.

The central processing unit 485 is also preferably configured to receive notifications from the hardware interface 495 when new devices are detected by the hardware interface. Hardware interface 495 can be a combination electromechanical detector with controlling software that communicates with the CPU 485 and interacts with new devices. The hardware interface 495 may be a firewire port, a USB port, a Bluetooth or infrared wireless unit, or any of a variety of wired or wireless access mechanisms. Examples of hardware that may be linked with the device 450 include data storage devices, computing devices, headphones, microphones, and the like.

FIG. 9 is a block diagram illustrating an exemplary computer system 550 that may be used in connection with the various embodiments described herein. For example, the computer system 550 may be used in conjunction with the tracking server 40 previously described with respect to FIGS. 3. However, other computer systems and/or architectures may be used, as will be clear to those skilled in the art.

The computer system 550 preferably includes one or more processors, such as processor 552. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 552.

The processor 552 is preferably connected to a communication bus 554. The communication bus 554 may include a data channel for facilitating information transfer between storage and other peripheral components of the computer system 550. The communication bus 554 further may provide a set of signals used for communication with the processor 552, including a data bus, address bus, and control bus (not shown). The communication bus 554 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

Computer system 550 preferably includes a main memory 556 and may also include a secondary memory 558. The main memory 556 provides storage of instructions and data for programs executing on the processor 552. The main memory 556 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 558 may optionally include a hard disk drive 560 and/or a removable storage drive 562, for example a floppy disk drive, a magnetic tape drive, a compact disc (“CD”) drive, a digital versatile disc (“DVD”) drive, etc. The removable storage drive 562 reads from and/or writes to a removable storage medium 564 in a well-known manner. Removable storage medium 564 may be, for example, a floppy disk, magnetic tape, CD, DVD, etc.

The removable storage medium 564 is preferably a computer readable medium having stored thereon computer executable code (i.e., software) and/or data. The computer software or data stored on the removable storage medium 564 is read into the computer system 550 as electrical communication signals 578.

In alternative embodiments, secondary memory 558 may include other similar means for allowing computer programs or other data or instructions to be loaded into the computer system 550. Such means may include, for example, an external storage medium 572 and an interface 570. Examples of external storage medium 572 may include an external hard disk drive or an external optical drive, or and external magneto-optical drive.

Other examples of secondary memory 558 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage units 572 and interfaces 570, which allow software and data to be transferred from the removable storage unit 572 to the computer system 550.

Computer system 550 may also include a communication interface 574. The communication interface 574 allows software and data to be transferred between computer system 550 and external devices (e.g. printers), networks, or information sources. For example, computer software or executable code may be transferred to computer system 550 from a network server via communication interface 574. Examples of communication interface 574 include a modem, a network interface card (“NIC”), a communications port, a PCMCIA slot and card, an infrared interface, and an IEEE 1394 fire-wire, just to name a few.

Communication interface 574 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 574 are generally in the form of electrical communication signals 578. These signals 578 are preferably provided to communication interface 574 via a communication channel 576. Communication channel 576 carries signals 578 and can be implemented using a variety of wired or wireless communication means including wire or cable, fiber optics, conventional phone line, cellular phone link, wireless data communication link, radio frequency (RF) link, or infrared link, just to name a few.

Computer executable code (i.e., computer programs or software) is stored in the main memory 556 and/or the secondary memory 558. Computer programs can also be received via communication interface 574 and stored in the main memory 556 and/or the secondary memory 558. Such computer programs, when executed, enable the computer system 550 to perform the various functions of the present invention as previously described.

In this description, the term “computer readable medium” is used to refer to any media used to provide computer executable code (e.g., software and computer programs) to the computer system 550. Examples of these media include main memory 556, secondary memory 558 (including hard disk drive 560, removable storage medium 564, and external storage medium 572), and any peripheral device communicatively coupled with communication interface 574 (including a network information server or other network device). These computer readable mediums are means for providing executable code, programming instructions, and software to the computer system 550.

In an embodiment that is implemented using software, the software may be stored on a computer readable medium and loaded into computer system 550 by way of removable storage drive 562, interface 570, or communication interface 574. In such an embodiment, the software is loaded into the computer system 550 in the form of electrical communication signals 578. The software, when executed by the processor 552, preferably causes the processor 552 to perform the inventive features and functions previously described herein.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A computer implemented method for tracking analyte measurements by a wireless measurement device communicatively coupled with a tracking server via a wireless communication network, comprising:
establishing a set of use parameters at the tracking server for the measurement device, the use parameters including an authorized number of uses;
receiving a measurement report from the measurement device via the wireless communication network;
adjusting the number of authorized uses in the use parameters for the measurement device; and
providing a notice when the number of authorized uses in the use parameters for the measurement device reaches a predetermined threshold.

2. The method of claim 1, wherein the providing step further comprises providing notice via the wireless communication network.

3. The method of claim 2, wherein the providing step further comprises providing the notice to the user.

4. The method of claim 2, wherein the providing step further comprises providing the notice to a party other than the user.

5. The method of claim 4, wherein the party other than the user is a medical professional.

6. The method of claim 4, wherein the party other than the user is an insurance company.

7. The method of claim 4, wherein the party other than the user is a member of the user's family.

8. The method of claim 1, wherein the measurement report comprises information about an analyte level for a user

9. The method of claim 1, wherein the adjusting step further comprises reducing the number of authorized uses for the measurement device.

10. The method of claim 1, wherein the adjusting step further comprises reducing the number of authorized uses for the user.

11. A system for tracking measurements by an analyte measurement device communicatively coupled with a tracking server via a wireless communication network, comprising:
a plurality of analyte measurement devices configured to measure an analyte level of a user;
a tracking server, communicatively coupled with the plurality of wireless measurement devices via a wireless communication network;
a communication module on the tracking server, the communication module configured to manage communications over the wireless communication network between an analyte measurement device and the tracking server;
a device module on the tracking server, the device module configured to send and receive data to and from an analyte measurement device in cooperation with the communication module, wherein the device module receives analyte measurement reports from an analyte measurement device and adjusts a number of authorized uses for the analyte measurement device and provides the adjusted number of authorized uses to the analyte measurement device in response.

12. The system of claim 11, wherein the device module is further configured to provide a notice when the number of authorized uses reaches a predetermined threshold.

13. The system of claim 12, wherein the device module is further configured to provide notice to the user.

14. The system of claim 12, wherein the device module is further configured to provide notice to a party other than the user.

15. The system of claim 14, wherein the party other than the user is a medical professional.

16. The system of claim 14, wherein the party other than the user is an insurance company.

17. The system of claim 14, wherein the party other than the user is a member of the user's family.

18. The system of claim 11, wherein the device module is further configured to de-authorize a particular analyte measurement device when the number of authorized uses for the particular analyte measurement device reaches zero.

19. The system of claim 11, further comprising a user module on the tracking server, the user module configured to maintain a number of authorized uses for a user and receive usage information for a user from the device module, adjust the number of authorized uses for the user, and provide the adjusted number of authorized uses to the device module in response.

20. The system of claim 19, wherein the user module is further configured to de-authorize a particular user when a number of authorized uses for the particular user reaches zero.

21. The system of claim 11, wherein the device module is further configured to provide a notice when the number of analyte measurement reports received from an analyte measurement device in a predetermined time period drops below a minimum number.
